(19) United States
(12) Patent Application Publication
(10) Pub. No.: US 2025/0342778 A1
(43) Pub. Date: Nov. 6, 2025

(54) BIDIRECTIONAL FEEDBACK SYSTEM AND RESPECTIVE METHOD

(71) Applicants: INSTITUTO PEDRO NUNES, ASSOCIAÇÃO PARA A INOVAÇÃO E DESENVOLVIMENTO EM CIÊNCIA E TECNOLOGIA, Coimbra (PT); Universidade de Coimbra, Coimbra (PT)

(72) Inventors: António Alberto LINDO JEGUNDO DA CUNHA, ALMALAGUÊS (PT); Rui Pedro DUARTE CORTESÃO, Coimbra (PT); João Manuel LEITÃO QUINTAS, ASSAFARGE (PT); Cristiana Filipa PINTO DA COSTA, LORVÃO (PT); Lúcia Margarida BAPTISTA DAS NE VES, Coimbra (PT); Guilherme Alexandre DA COSTA CORREIA, GRÂNDOLA (PT); Sérgio MARTINS DE SOUSA, ROMÃS (PT); Vanessa Susana GONÇALVES DA CUNHA, Coimbra (PT)

(73) Assignees: INSTITUTO PEDRO NUNES, ASSOCIAÇÃO PARA A INOVAÇÃO E DESENVOLVIMENTO EM CIÊNCIA E TECNOLOGIA, Coimbra (PT); Universidade de Coimbra, Coimbra (PT)

(21) Appl. No.: 18/869,875

(22) PCT Filed: May 26, 2023
(86) PCT No.: PCT/IB2023/055426

§ 371 Date: Nov. 27, 2024

(30) Foreign Application Priority Data
May 27, 2022 (PT) 118014

(51) Int. Cl. G09B 23/28; A61B 8/00

(52) U.S. Cl. CPC G09B 23/28; A61B 8/4218; A61B 8/4263; A61B 8/462; A61B 8/464; A61B 8/469; A61B 8/565

(57) ABSTRACT

A bidirectional feedback system for remote spatial positioning correction of a robotic arm for ultrasound scanning is disclosed. An embodiment includes: a first robotic arm for ultrasound scanning; a second robotic arm for mirroring the first robotic arm; a first and second display; an electronic data processor configured for: receiving ultrasound scan images; sending the received ultrasound scan images to the two displays; mirroring the relative spatial positions of the first and second robotic arm, wherein: sensing a first relative spatial position from the first robotic arm, and moving the second robotic arm to the first relative spatial position; has higher priority than: sensing a second relative spatial position from the second robotic arm, and moving the first robotic arm to the second relative spatial position. Also disclosed is a respective method and use of said system for remote hands-on training, preferably for medical training, more preferably for ultrasound training.

### TECHNICAL FIELD

The present disclosure refers to a bidirectional feedback system for remote spatial positioning correction of a robotic arm for ultrasound scanning. For example, for delivering human-to-human distance and practical training, further providing real-time multimodal feedback, using haptic, visual and audio interaction interfaces.

### BACKGROUND

The document WO2015191910 discloses a method for reinforcing programming education through toy robot feedback, including: at a user device, remote from the toy robot: receiving a series of programming inputs from a user at a programming interface application on the user device; receiving a set of sensor measurements from the toy robot; automatically generating a set of control instructions for the toy robot based on a programming input of the series and the set of sensor measurements.

The document CN107263449A discloses a robot remote teaching system based on virtual reality.

The document US20,160,96270 discloses a robotic device may be operated by a learning controller comprising a feature learning configured to determine control signal based on sensory input.

The article “Twin Kinematics Approach for Robotic-Assisted Tele-Echography” details a control architecture for the robotic tele-echography system that allows the follower robot to act according to the commands of the leader robot with force sensation on the leader side in operation scenarios in environments with communication channels with small time delay.

These facts are disclosed in order to illustrate the technical problem addressed by the present disclosure.

### GENERAL DESCRIPTION

The present document discloses a bidirectional feedback system for remote spatial positioning correction of a robotic arm for ultrasound scanning comprising: a first robotic arm for ultrasound scanning comprising an end effector for the displacement of the first robotic arm by a first user; a second robotic arm for mirroring the first robotic arm comprising at least one handle for the displacement of the second robotic arm by a second user; a first and second display for displaying the ultrasound scanning images to the first and second users, respectively; an electronic data processor configured for: receiving ultrasound scan images corresponding to the spatial positioning and orientation of the end effector; sending the received ultrasound scan images to the two displays; mirroring the relative spatial positions of the first and second robotic arm, wherein: sensing a first relative spatial position from the first robotic arm, and moving the second robotic arm to the first relative spatial position; has higher priority than: sensing a second relative spatial position from the second robotic arm, and moving the first robotic arm to the second relative spatial position.

In an embodiment, a first user interacts with the first robotic arm by displacing the end effector, thus provide haptic feedback when moving to the second relative spatial position.

In an embodiment, the first robotic arm is configured to move freely, i.e., without physical resistance, when not receiving a second relative spatial position.

In an embodiment, the system further comprising a first set of cameras for recording the first user and/or the position of the first robotic arm, and a second set of cameras for recording the second user and/or the position of the second robotic arm, preferably a set of four cameras, for video communication between the first and second users.

In an embodiment, one of the two displays is configured for displaying the ultrasound scanning images, the first user images, the position of the first robotic arm, or a combination of these, to the second user.

In an embodiment, the system further comprising a first microphone and speaker, and second microphone and speaker for voice communication between the first and second users.

In an embodiment, the end effector is an ultrasound scanning probe.

In an embodiment, the first robotic arm further comprises a switch, preferably a foot switch, to turn on/off the spatial position input from the second user.

In an embodiment, the first and second displays are head mounted devices (HMD) to deliver extended reality (XR) interfaces, preferably for the first user and the second user.

In an embodiment, the system further comprising a keyboard and/or a mouse to input at least one annotation and/or a pointer position from the second user on the received ultrasound scan images.

In an embodiment, the second display is a touchscreen for the second user interact with, e.g., pointing on and/or taking notes.

In an embodiment, the first robotic arm and the second robotic arm are connected to the electronic data processor via a wireless internet connection, preferably a cellular connection, most preferably a 5G connection, or a Wi-Fi connection, or a satellite internet connection.

It is also disclosed the use of said system for remote hands-on training, preferably for medical training, more preferably for ultrasound training.

It is further disclosed a method of operation of a bidirectional feedback system for remote spatial positioning correction of a robotic arm for ultrasound scanning comprising the steps: receiving ultrasound scan images corresponding to the spatial positioning and orientation of the end effector; sending the received ultrasound scan images to the two displays; mirroring the relative spatial positions of the first and second robotic arm, wherein: sensing a first relative spatial position from the first robotic arm, and moving the second robotic arm to the first relative spatial position; has higher priority than: sensing a second relative spatial position from the second robotic arm, and moving the first robotic arm to the second relative spatial position.

In an embodiment, when moving the first robotic arm to the second spatial position the end effector provides force feedback to the first user and/or to the second user.

In an embodiment, the method further comprising the step of displaying the ultrasound scanning images, the first user recording, the position of the first robotic arm, or a combination of these, into a display to the first and/or second user.

In an embodiment, the method further comprising the step of receiving at least one annotation and/or a pointer position from the second user on the received ultrasound scan images and, displaying the at least one annotation and/or the pointer position on the first display to a first user.

### BRIEF DESCRIPTION OF THE DRAWINGS

The following figures provide preferred embodiments for illustrating the disclosure and should not be seen as limiting the scope of invention.

FIG. 1: Schematic representation of an embodiment of a bidirectional feedback system for remote spatial positioning correction of a robotic arm.

FIG. 2: Schematic representation of an embodiment of a trainee system part.

FIG. 3: Schematic representation of an embodiment of a system comprising two main parts, one actuated by a trainer and another by a trainee.

FIG. 4: Schematic representation of an embodiment of a bidirectional feedback system.

FIG. 5: Schematic representation of an embodiment of a bidirectional feedback system comprising a virtual reality set.

FIG. 6A, 6B, 6C: Schematic representation of an embodiment of a bidirectional feedback system assembled for a trainee interaction.

FIG. 7: Schematic representation of an embodiment of a bidirectional feedback system assembled for a trainer interaction.

FIGS. 8A, 8B, 8C: Flowchart representations of an embodiment of a communication between a trainer and a trainee.

FIG. 9: Flowchart representation of an embodiment of a method of operation of the bidirectional feedback system.

### DETAILED DESCRIPTION

The present document discloses a bidirectional feedback system for remote spatial positioning correction of a robotic arm for ultrasound scanning comprising: a first robotic arm for ultrasound scanning comprising an end effector for the displacement of the first robotic arm by a first user; a second robotic arm for mirroring the first robotic arm comprising at least one handle for the displacement of the second robotic arm by a second user; a first and second display for displaying the ultrasound scanning images to the first and second users, respectively; an electronic data processor configured for: receiving ultrasound scan images corresponding to the spatial positioning and orientation of the end effector; sending the received ultrasound scan images to the two displays; mirroring the relative spatial positions of the first and second robotic arm, wherein: sensing a first relative spatial position from the first robotic arm, and moving the second robotic arm to the first relative spatial position; has higher priority than: sensing a second relative spatial position from the second robotic arm, and moving the first robotic arm to the second relative spatial position. It is also disclosed a respective method and use of said system for remote hands-on training, preferably for medical training, more preferably for ultrasound training.

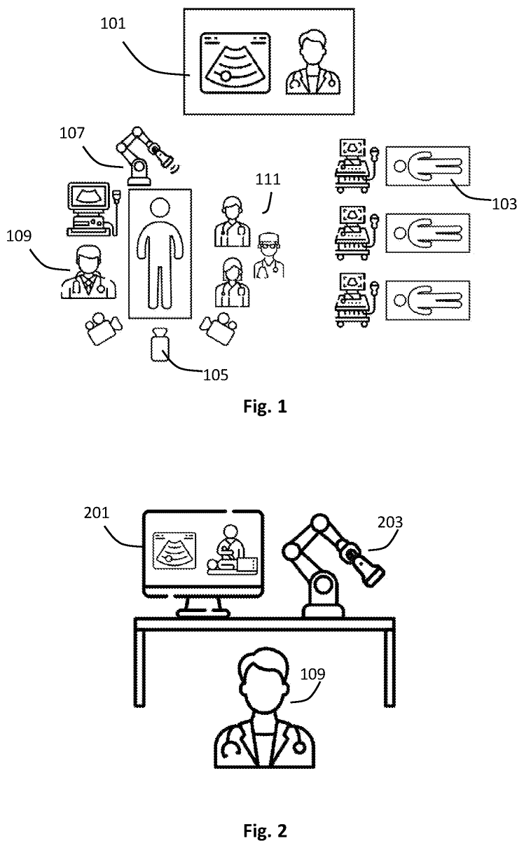

FIG. 1 shows a schematic representation of an embodiment of a bidirectional feedback system for remote spatial positioning correction of a robotic arm, wherein 101 represents a display with graphical interface, 103 represents a subject, 105 represents a camera, 107 represents a first robotic arm, 109 represents a trainer, and 111 represents a trainee.

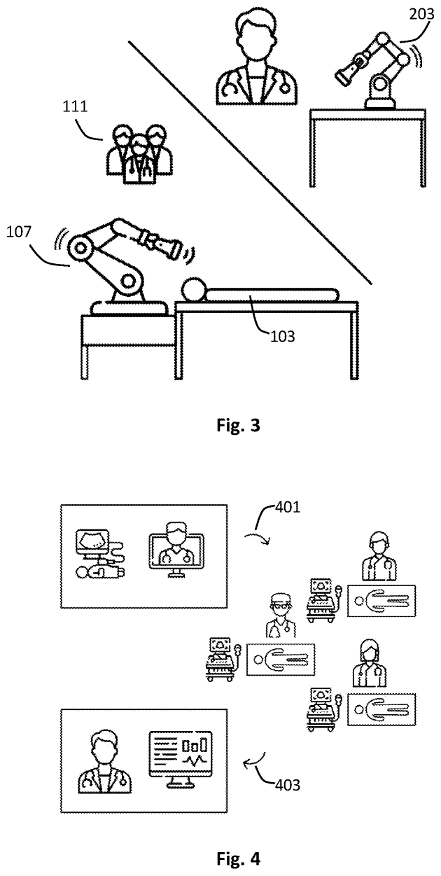

FIG. 2 shows a schematic representation of an embodiment of a trainee system part, wherein 109 represents a trainer, 201 represents an interface, and 203 represents a second robotic arm.

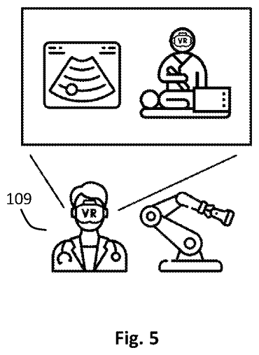

FIG. 3 shows a schematic representation of an embodiment of a system comprising two main parts, one actuated by a trainer and another by a trainee, wherein 103 represents a subject, 107 represents a first robotic arm, 109 represents, 111 represents a trainee, and 203 represents a second robotic arm.

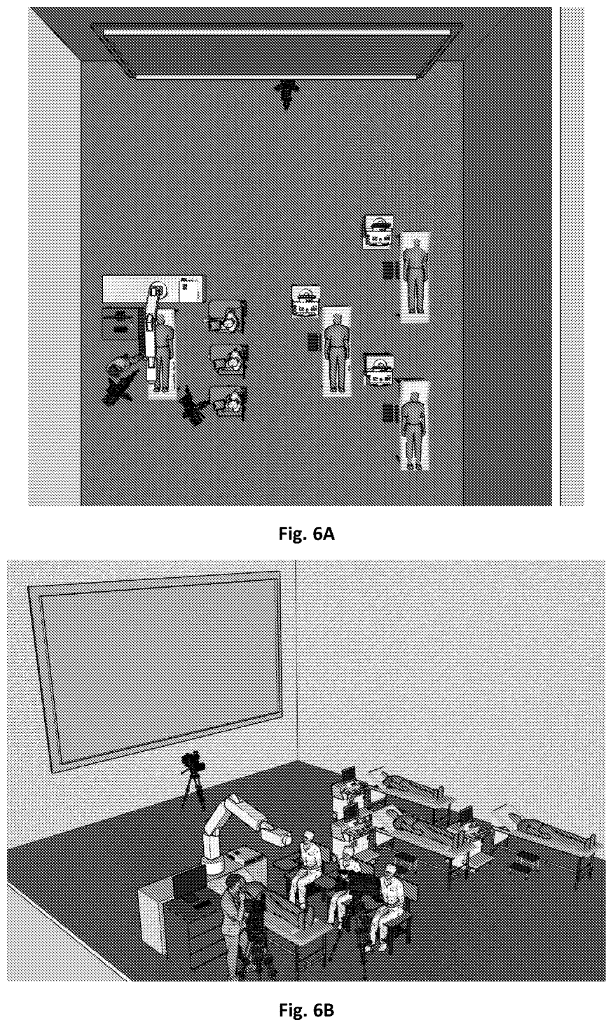

FIG. 4 shows a schematic representation of an embodiment of a bidirectional feedback system, wherein 401 represents data sharing and information sharing, and 403 represents the collection of data.

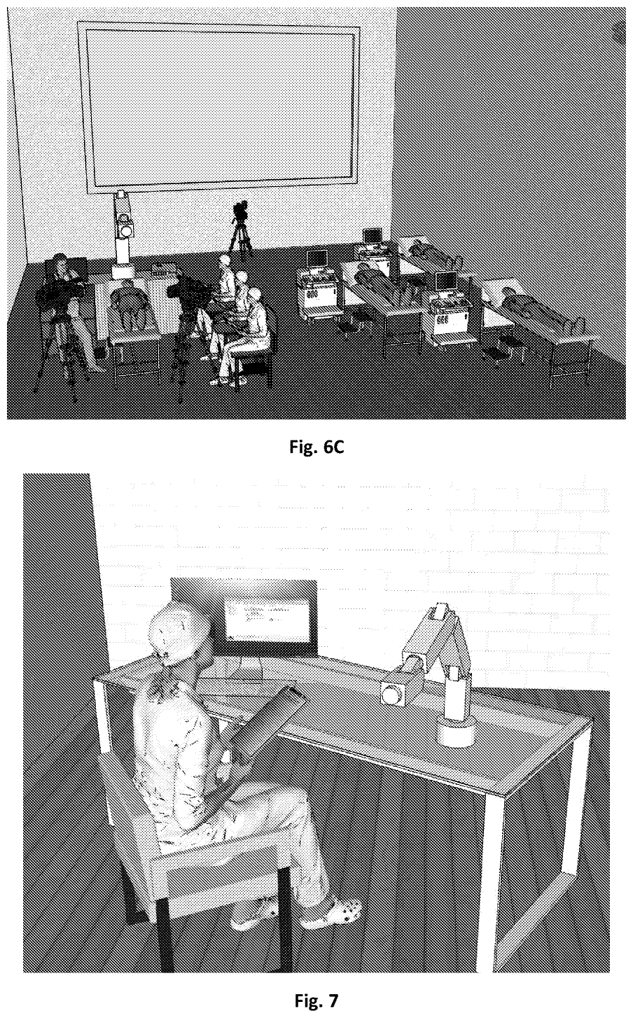

FIG. 5 shows a schematic representation of an embodiment of a bidirectional feedback system comprising a virtual reality set, wherein 109 represents a trainer.

In an embodiment, only the trainer uses the VR set, since the trainee is physically in the same place as the subject. The immersive functionality is required at the trainer station, when the trainer is performing a tele-operation task.

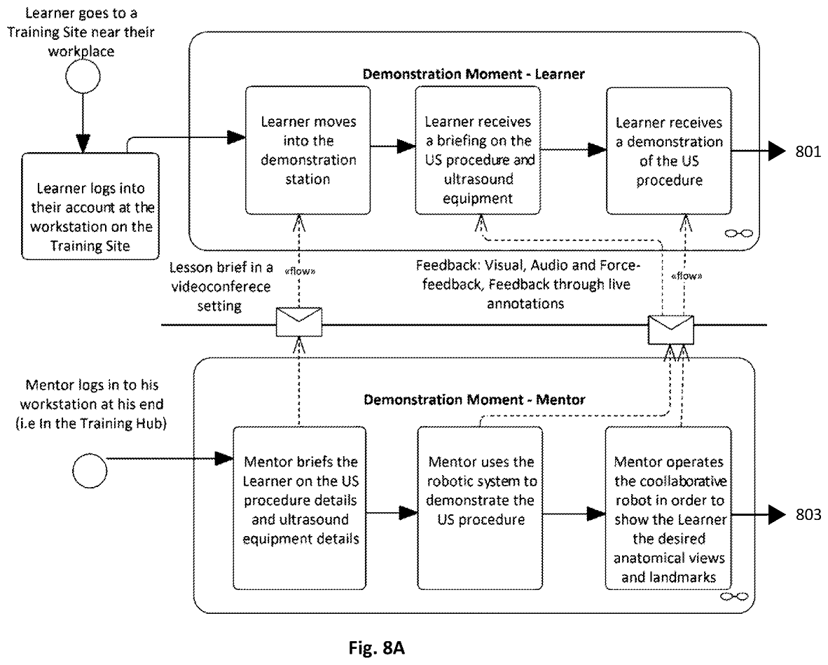

FIG. 6A, 6B, 6C shows a schematic representation of an embodiment of a bidirectional feedback system assembled for a trainee interaction.

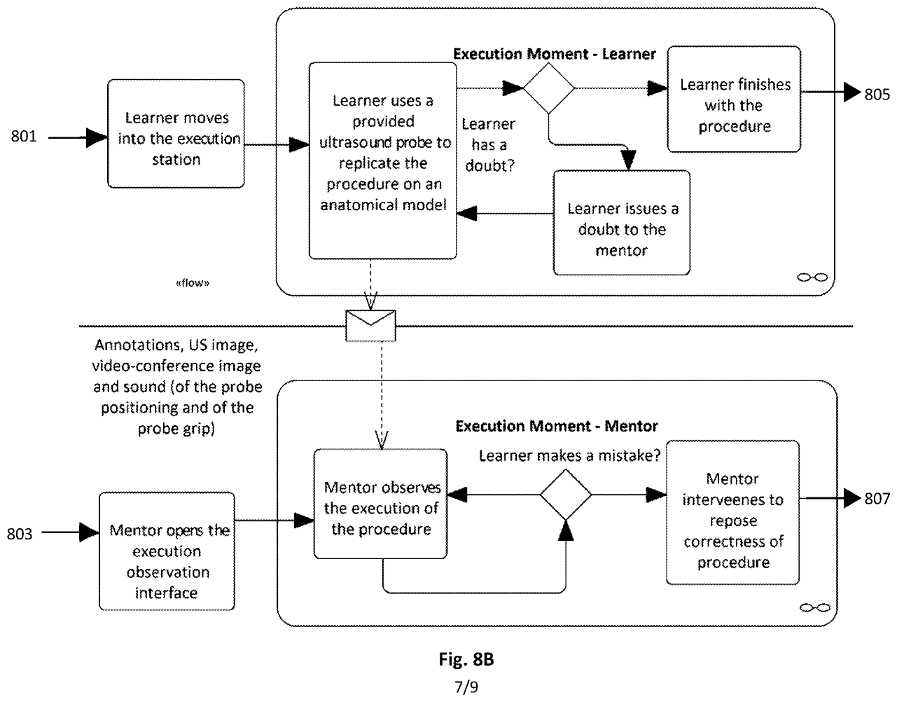

FIG. 7 shows a schematic representation of an embodiment of a bidirectional feedback system assembled for a trainer interaction.

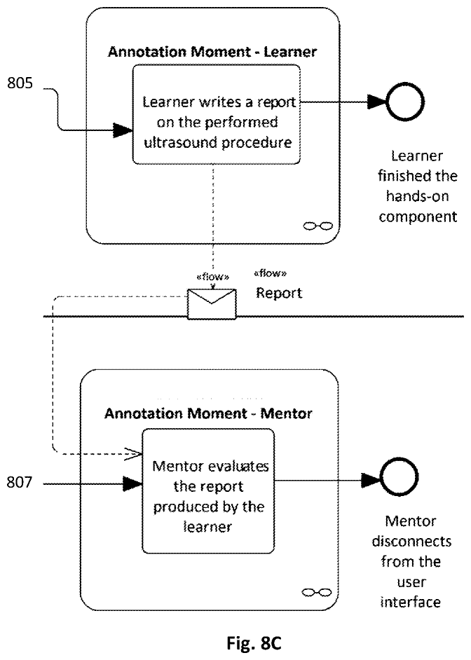

FIGS. 8A, 8B, 8C show flowchart representations of an embodiment of a communication between a trainer and a trainee, wherein 801 represents an output from a learner on a demonstration moment, 803 represents an output from a mentor on a demonstration moment, 805 represents an output from a learner on an execution moment, and 807 represents an output from a mentor on an execution moment.

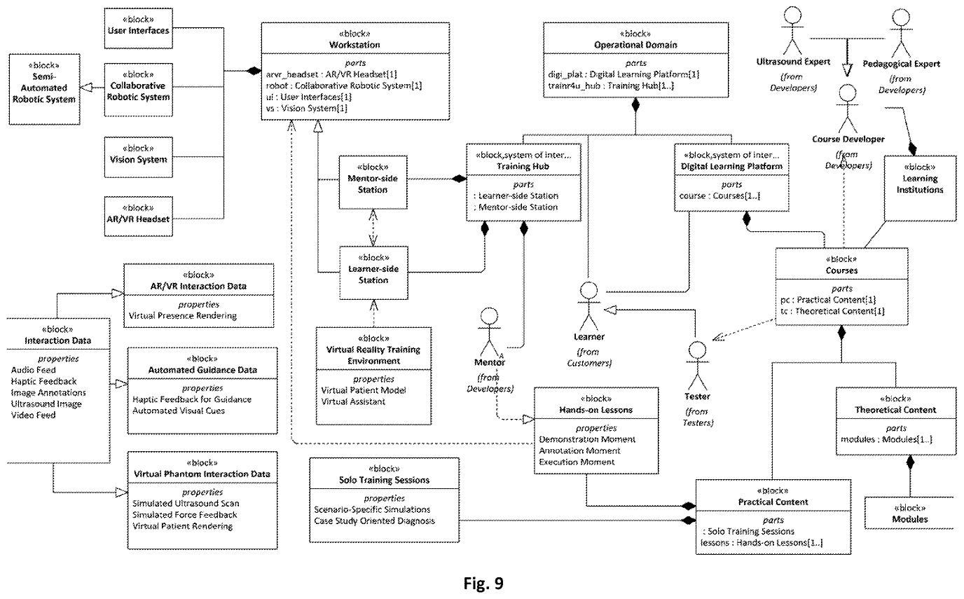

FIG. 9 shows a flowchart representation of an embodiment of a method of operation of the bidirectional feedback system.

This system and method can be applied to share human-to-human practical skills at a distance, namely to train remotely a user for performing ultrasound scanning.

In one embodiment, a system and method of online training through an e-learning platform, combining video and audio with remote hands-on training with haptic feedback, with robotic assistance for the performance of practical training exercises, is disclosed.

In one embodiment, the e-learning platform is used for distance learning and hands-on training so the trainee can feel, preferably in real-time, the haptic feedback and, thus, the spatial positioning correction performed by the trainer while the trainee performs physical operations using an end effector, irrespective of the physical distance between the trainer and the trainee.

In one embodiment, the system allows three different types of feedback: visual, audio and haptic.

In one embodiment, the system is assembled in two different locations, also called stations, namely a trainer station and a trainee station, connected to each other through an Internet connection to enhance the above-mentioned feedbacks.

In one embodiment, the trainee station comprises a robotic arm equipped with an end effector, allowing the haptic feedback between the trainer and the trainee, a computer, and a display to visualise the image collected and to act as an interface with the trainer, with a webcam for visual and audio interaction (video and audio feedback).

In one embodiment, the trainer station, which is located remotely, comprises a robotic arm with force-feedback capability, which allows controlling the position and orientation of the robot's end-effector positioned in the trainee station.

In one embodiment, the haptic device is the robotic arm, from both the trainee Station and the trainer Station, allowing that the movements performed by one side are mimicked by the other. This behaviour allows the trainer to teach, guide and correct the movements of the trainee.

In one embodiment, the trainer station is composed of a computer, equipped with a webcam, microphone and speakers together with a robotic arm that provides a haptic interface. Through the trainer station, the trainer can control any device being manipulated by the trainee using the trainee station, allowing their mutual deeper immersion. Moreover, this solution allows the trainer to monitor and control any performance of the trainee, further observing, pointing or highlighting the ultrasound image collected in real-time and also see the trainees while they are performing the exercises on their station.

In one embodiment, the trainer station comprises a robotic arm with equivalent technical capabilities, for example a robotic arm that is a scaled down version of the trainee robotic arm, to the one embedded on the trainee station, allowing the co-manipulation of any device under the scope of the training session.

In another embodiment, to provide a deeper immersion of the trainer in the trainees' station, the system has the capability to add Extended Reality into the co-manipulation of the robot. Accordingly, the trainer sees the robot as his/her own arm and hand, thus interacting with the devices and the trainees, teaching, guiding and correcting them, when needed.

Since both trainer robotic arms and trainee robotic arms have a similar geometric structure, the teleoperation architecture follows a position-position approach in the joint space. The trainee robotic arms reference is given by the trainer robotic arms joint positions, while trainer robotic arms input is the torque computed by the trainee robotic arms joint position controller.

In an embodiment, the trainer has access to a virtual pointer that is shown on top of the ultrasound scan images. The pointer is activated whenever the trainer moves his/her finger, for touchscreens, or right-click mouse over the ultrasound scan images. Then, those positions are sent, separately, to the trainee's station, where the position is shown. The position is mapped considering the image display device of the trainee station (resolution, size).

Additionally, the system is capable of:
Automatic evaluation of the quality of ultrasound images (namely the signal-to-noise ratio) acquired in real-time, generating feedback from the image that is used to automatically adjust the robot's control; including medical image processing and segmentation;
Automatically reconstruct 3D volumes from 2D ultrasound images acquired by controlled rotational movements of the probe; and
Support the trainer to faster recognize the anatomic images and increase the quality of diagnosis with US (ultrasound) images as well as mitigate the issue of operator-dependent diagnosis variability and enable coverage of geographically remote areas.

In one embodiment, the trainee station is composed by: 1) An ultrasound probe connected to a computer which provides the Graphical User Interface to configure the probe pre-sets and mediates the collection of the ultrasound images. The same computer also provides a video conference to communicate with the trainer station, composed by at least 4 cameras, one facing the trainee while performing the ultrasound exercise and the remaining cameras facing the robotic arm and the trainee performing the ultrasound exercise; 2) A robotic arm system coupled to an ultrasound probe, the latter acting as end effector, allowing its control by the trainee directly in his/her station or remotely by the trainer through the trainer station; and 3) An extended reality system, including a phantom model of the human body to simulate clinical scenarios during the teaching/training activities.

In the same particular embodiment, the trainer station comprises: 1) A replica of the robotic arm, connected to a computer that manages the control and the communication with the trainee station; 2) A Graphical User Interface where the trainer can control the ultrasound image presented, further allowing the annotation, pointing and contents sharing with the trainee station; 3) An interface which communicates with RIS and PACS systems through the protocols HL7 and DICOM and save ultrasound images and also communicate with the trainee, optionally the trainer has a pointer that points in the ultrasound image for the trainee to see in real time in the trainee station; 4) Video conference means, including a camera facing the trainer, together with audio facility; 5) An Extended Reality interface to provide a deeper immersion of the trainer in the trainees' station, enhancing the co-manipulation of the ultrasound probe.

The Graphical User Interface (GUI) allows the communication between the two stations and the configuration of the probe presets. The GUI include features to authenticating of the users, load of worklists, display ultrasound images and the correspondent controllers to changes the presets, save images and take annotation on it. The ultrasound image collected on the trainee station will be shared with the trainer Station, allowing to the trainer to point, analyse and give feedback on the anatomic ultrasound image collected by the trainee.

The stations are connected through an internet connection, including 5G, through a dedicated VPN, and a graphical interface that allows monitoring, controlling and evaluating the procedure during the lecture.

As advantages of the disclosed embodiments, it is worth mentioning namely the chance to learn from specialists without major travels and costs. The trainer could teach without leaving his/her office like he/she was holding the trainee hand and that trainee could be in a remote location. Neither of them has to spend days and money travelling because the course goes to them.

The present disclosure allows bilateral control of the system, enabling, for example, in a classroom operation scenario, a mentor and a student to develop tasks and learning in co-manipulation. In this scenario, the mentor's station has priority over the student's, allowing, however, that both the student and the mentor maintain cooperation processes that do not compromise the integrity and stability of the system. This priority is due to the necessary force scaling needed to make the robotic systems at each end of the system compatible, in terms of operational payload. The term “comprising” whenever used in this document is intended to indicate the presence of stated features, integers, steps, components, but not to preclude the presence or addition of one or more other features, integers, steps, components, or groups thereof.

The disclosure should not be seen in any way restricted to the embodiments described and a person with ordinary skill in the art will foresee many possibilities to modifications thereof. The above-described embodiments are combinable.

The following claims further set out particular embodiments of the disclosure.

1. A bidirectional feedback system for remote spatial positioning correction of a robotic arm for ultrasound scanning, comprising:
a first robotic arm for ultrasound scanning comprising an end effector for the displacement of the first robotic arm by a first user;
a second robotic arm for mirroring the first robotic arm comprising at least one handle for the displacement of the second robotic arm by a second user;
a first and second display for displaying the ultrasound scanning images to the first and second users, respectively; and
an electronic data processor configured to:
receive ultrasound scan images corresponding to the spatial positioning and orientation of the end effector;
send the received ultrasound scan images to the two displays;
mirror the relative spatial positions of the first and second robotic arm,
wherein: sensing a first relative spatial position from the first robotic arm, and moving the second robotic arm to the first relative spatial position have a higher priority than sensing a second relative spatial position from the second robotic arm and moving the first robotic arm to the second relative spatial position.

2. The system according to claim 1, further comprising a first set of cameras for recording the first user and/or the position of the first robotic arm, and a second set of cameras for recording the second user and/or the position of the second robotic arm.

3. The system according to claim 1, wherein one of the two displays is configured for displaying the ultrasound scanning images, the first user images, the position of the first robotic arm, or a combination of these, to the second user.

4. The system according to claim 1, further comprising a first microphone and speaker, and second microphone and speaker for voice communication between the first and second users.

5. The system according to claim 1, wherein the end effector is an ultrasound scanning probe.

6. The system according to claim 1, wherein the first robotic arm further comprises a switch, preferably a foot switch, to turn on/off the spatial position input from the second user.

7. The system according to claim 1, wherein the first and second displays are head mounted devices (HMD) to deliver extended reality (XR) interfaces.

8. The system according to claim 1, further comprising a keyboard and/or a mouse to input at least one annotation and/or a pointer position from the second user on the received ultrasound scan images.

9. The system according to claim 1, wherein the second display is a touchscreen for the second user interact with.

10. The system according to claim 1, wherein the first robotic arm and the second robotic arm are connected to the electronic data processor via a wireless internet connection.

11. A method of remote hands-on user_training comprising use of the system according to claim 1.

12. A method of operation of a bidirectional feedback system for remote spatial positioning correction of a robotic arm for ultrasound scanning, comprising the steps of:
receiving ultrasound scan images corresponding to the spatial positioning and orientation of the end effector;
sending the received ultrasound scan images to the two displays; and
mirroring the relative spatial positions of the first and second robotic arm, wherein sensing a first relative spatial position from the first robotic arm and moving the second robotic arm to the first relative spatial position have a higher priority than sensing a second relative spatial position from the second robotic arm, and moving the first robotic arm to the second relative spatial position.

13. The method according to claim 12, further comprising the step of displaying the ultrasound scanning images, the first user recording, the position of the first robotic arm, or a combination of these, into a display to the first and/or second user.

14. The method according to claim 12, further comprising the steps of
receiving at least one annotation and/or a pointer position from the second user on the received ultrasound scan images; and,
displaying the at least one annotation and/or the pointer position on the first display to a first user.

15. The method of claim 12, further comprising the steps of:
sensing the first relative spatial position from the first robotic arm;
moving the second robotic arm to the first relative spatial position;
sensing the second relative spatial position from the second robotic arm; and
moving the first robotic arm to the second relative spatial position.

16. The system of claim 1, wherein the electronic data processor is further configured to:
sense the first relative spatial position from the first robotic arm;
move the second robotic arm to the first relative spatial position;
sense the second relative spatial position from the second robotic arm; and
move the first robotic arm to the second relative spatial position.
